(19) United States
(12) Patent Application Publication
(10) Pub. No.: US 2004/0208784 A1
(43) Pub. Date: Oct. 21, 2004

(54) Sterilization processor used for operation of the sterilization art and method of infectivity drainage

(75) Inventors: Tsutomu Matsuda, Tokyo (JP); Nobuyuki Machida, Tokyo (JP); Yuji Arai, Tokyo (JP); Yoshiaki Itoh, Tokyo (JP)

Correspondence Address: HAUPTMAN KANESAKA BERNER PATENT AGENTS, LLP, Suite 310, 1700 Diagonal Road, Alexandria, VA, 22314, US

(73) Assignee: NEC AMENIPLANTEX, LTD.

(21) Appl. No.: 10/774,439

(22) Filed: Feb. 10, 2004

(30) Foreign Application Priority Data
Apr. 17, 2003 (JP) 2003-113217
Apr. 17, 2003 (JP) 2003-113218
Apr. 17, 2003 (JP) 2003-113219

(51) Int. Cl. A61L 2/04

(52) U.S. Cl. 422/38; 422/307

(57) ABSTRACT

Method for sterilizing infectious waste water and a system thereof disclosed in the present invention comprise pump feeding step A, heating/sterilization step B, drainage step C, cleaning step D and vacuum suction step E. The pump feeding step A is a step wherein infectious waste water in a raw water tank is pumped to a tank body of a sterilization tank, the heating/sterilization step B is a step wherein infectious waste water pooled into the tank body is indirectly heated to sterilize the infectious waste water, the drainage step C is a step in which waste water treated by heating/sterilization is discharged from the tank body, the cleaning step D is a step in which after the drainage step, washing water is showered to clean the tank body. The vacuum suction step E is a step wherein infectious waste water in a raw water tank is vacuum-suctioned, without using a vacuum pump, into the tank body which is rendered negative in pressure by the cleaning step, and used in place of the pump feeding step A or in combination with the pump feeding step A.

### BACKGROUND OF THE INVENTION

1. Field of the Invention

This invention relates to a method for sterilizing infectious waste water discharged from health care facilities such as hospitals and a system thereof.

2. Description of the Related Art

Waste water discharged from infectious disease wards or from dissection rooms at health care facilities (hereinafter referred to as infectious waste water) contains blood or body fluids into which pathogenic microorganisms may be contained. Therefore, sufficient sterilization should be exercised in discharging the abovementioned water.

A steam heat sterilization method is a typical method for sterilizing infectious waste water discharged from health care facilities. In the steam heat sterilization method, infectious waste water pooled into a raw water tank on the premises is pumped up by using a submerged pump, transferred into a sterilization tank, and steam is directly fed into the infectious waste water in the sterilization tank, thus allowing the infectious waste water to be exposed to high-temperature steam for a certain period of time to effect sterilization. Unlike incineration, the steam heat sterilization method is free of possible risk of gaseous outflow or dioxin production, thus removing the necessity for particularly troublesome post-procedures, except for lowering the temperature of the waste water to less than a certain level prior to discharge of the waste water.

Since this sterilization is to allow infectious waste water to be exposed directly to high-temperature steam, it is called the direct heating sterilization method. Waste water after sterilization is transferred into a cooling tank by opening a valve mounted at the bottom of the sterilization tank, pooled temporarily in the cooling tank, mixed with city water as cooling water and released into the sewage system after being lowered down to a normal temperature (about 40° C.). Since the direct heating sterilization method is to effect sterilization by allowing the heat of the steam directly provided into infectious waste water in a sterilization tank to directly act on infectious microorganisms in waste water, it was considered to be excellent in sterilization effects.

However, the direct heating sterilization method has been found to have the following problems: namely, in employing this sterilization method, direct heating can be applied to waste water only in a limited area, or in the vicinity of steam pipes and a larger sterilization tank may cause an area not to be sufficiently heated, thus resulting in a decrease in sterilization efficiency. More particularly, steam is ejected from a steam pipe inserted into the surface of infectious waste water from the sterilization tank above, thus giving heat only to the waste water coming into contact with the steam while the ejected steam rises to the surface.

In order to resolve these problems, a method for sterilizing infectious waste water and a system thereof have been developed, by which infectious waste water contained in a sterilization tank is uniformly heated to carry out an effective sterilization (refer to patent literature 1). Patent literature 1. Japanese Published Unexamined Patent Application No. 2003-53326

The method for sterilizing infectious waste water as set forth in the patent literature 1 is a method for sterilizing infectious waste water having a sterilization tank on the basis of indirect heating, the method comprising the steps of vacuum water supply, heating/sterilization, and drainage, more particularly, the vacuum water supply step is a step wherein deaeration is effected inside a tank body of a sterilization tank by using a vacuum pump, thus vacuum suctioning infectious waste water into the tank body, the heating/sterilization is a step wherein the heat of the steam is allowed to act on infectious waste water through the wall surface of the tank body, thus sterilizing infectious waste water, and the drainage step is a step wherein heated and sterilized waste water is discharged from the tank body.

According to the method for sterilizing infectious waste water as set forth in the patent literature 1, heat convection develops in infectious waste water contained in a tank body, providing uniform heating to the whole of the infectious waste water to attain an effective sterilization, and infectious waste water contained in a waste water pit is vacuum-suctioned and transferred to the tank body, thereby preventing contamination of a vacuum pump and pump pipes to attain an easier maintenance after work. In the actual step, which is not described in the patent literature 1, the tank body is cleaned by showering washing water after the drainage step, and then the vacuum water supply step is carried out from the beginning. Thus, the cleaning step prior to the vacuum water supply step produces a negative pressure inside the tank body, which may be utilized in suctioning the infectious waste water into the tank body.

Further, as shown in FIG. 3(b), the sterilization tank 31 to be used in the system for sterilizing infectious waste water is provided with the tank body 32 and the steam heating means 33. The tank body 32 is a tank for receiving infectious waste water to be sterilized, the steam heating means 33 consists of steam generator 34 and heating part 35, and heating part 35 is assembled on the outer wall of the tank body 32, receiving steam which is produced by the steam generator 34 and allowing the heat of the steam to act on infectious waste water contained in the tank body 32. The infectious waste water, which constitutes water level WL within a range of the height H1 of heating part 35, is fed into the tank body 32, and the tank body 32 is heated from the surrounding surface, thus providing heat convection to the infectious waste water to attain a uniform heating and sterilization. The numeral 36 denotes a temperature sensor.

Infectious waste water discharged from health care facilities is rich in solids such as blood, protein, and fat. When waste water is heated by steam through the wall surface of the tank body 32, solids such as blood, protein, and fat are precipitated on the tank wall, or attached on the tank wall and burnt thereon, which poses problems. Once the solids are attached as scales on the wall of the tank, they are not easily separated or removed. If the solids remain without attention being paid, they may corrode the wall surface of the tank body. On the other hand, if the solids are separated, they may pose a problem of blocking the pipes for feeding treated waste water from the tank body 32. Therefore, it has been explained that the wall surface of the tank body 32 must be cleaned frequently.

In addition, in the system for sterilizing infectious waste water as set forth in the patent literature 1, the infectious waste water sterilized in the tank body 32 is released into the sewage system through the drain pipe 37, and the waste water heated to a high temperature by the sterilization step is mixed with cooling water to lower the temperature to an acceptable level (for example, 40° C. to 45° C.) before release into the sewage system.

As shown in FIG. 6, in the cooling step, the drain pipe 37 is provided with the cooling tank 38, the treated waste water contained in the tank body 32 is transferred and pooled into the cooling tank 38, city water 39 is fed as cooling water to the treated waste water pooled into the cooling tank 38 to effect cooling, and released into the sewage pipe 40. It was found that abnormal vibrations and noises developed in the cooling tank 38, when city water 39 was fed to effect cooling.

It is still not clear why the cooling step causes these vibrations and noises. Since infectious waste water must be treated at temperatures from 121° C. to 134° C., treated waste water pooled into the cooling tank 38 includes steam, the temperature of which exceeds 100° C., maintaining a high temperature, although the temperature is lowered to some extent. Thus, it is likely that a steam explosion-related expansion when city water is fed into a large amount of high-temperature waste water and subsequent abrupt-cooling related shrinkage are repeated to develop abnormal vibrations and noises.

When vibrations are provided to the cooling tank 38, there is a problem wherein cracks may develop on welded parts or threaded parts of the cooling tank 38, thus resulting in leakage of waste water. Further, as a matter of course, noises cause a public nuisance in the vicinity of the system. For drainage, treated waste water must be forcibly discharged due to negative pressure in the tank body. In the system for sterilizing infectious waste water as set forth in the patent literature 1, an example is shown wherein high-pressure air generated by a compressor is blown into a tank body to forcibly discharge the treated waste water.

The object of the invention is to provide a method for sterilizing infectious waste water and a system thereof in which negative pressure produced inside a tank body after the cleaning step is utilized to suction infectious waste water pooled into a raw water tank into the tank body, thus reducing running costs, actual problems found in running a sterilization system based on indirect heating are resolved, and treated infectious waste water is cooled and released into the sewage system, without allowing solids in the infectious waste water to attach onto an inner wall of the tank body or causing vibrations and noises.

### SUMMARY OF THE INVENTION

In order to achieve the above object, the method for sterilizing infectious waste water according to the present invention is a method for sterilizing infectious waste water, comprising the steps of water supply, heating/sterilization, drainage, and cleaning, wherein,

the water supply step consists of the pump feeding step and vacuum suction step,

the pump feeding step is a step wherein infectious waste water in a raw water tank is pumped to a tank body,

the vacuum suction step is a step wherein infectious waste water remaining in a raw water tank after the cleaning step or infectious waste water newly pooled into the raw water tank is vacuum-suctioned into the tank body which is rendered negative in pressure by the cleaning step, and used in place of the pump feeding step or in combination with the pump feeding step,

the heating/sterilization step is a step wherein the heat of the steam is passed through the wall surface of the tank body, allowed to act on infectious waste water suctioned into the tank body of a sterilization tank, thus attaining sterilization of the infectious waste water,

the drainage step is a step wherein heated and sterilized waste water is discharged from the tank body,

the cleaning step is a step wherein washing water is showered to the tank body of the sterilization tank after the drainage step to clean the tank body.

Further, the pump feeding step is used in suctioning for the first time infectious waste water pooled into a raw water tank into the tank body of the sterilization tank,

the vacuum suction step is a step wherein after the pump feeding step, heating/sterilization step, drainage step and cleaning step are carried out and then infectious waste water pooled into the raw water tank is suctioned into the tank body which is rendered negative in pressure by the cleaning step.

The invention also provides a method for sterilizing infectious waste water in a sterilization tank on the basis of indirect heating, the method comprising steps of water supply, heating/sterilization, and drainage, in which

the sterilization tank consists of a tank body receiving infectious waste water and a heating part that steam-heats the tank body externally,

the water supply step is a step wherein infectious waste water is supplied into the tank body either by a pump feeding step or vacuum suction step,

the pump feeding step is a step wherein infectious waste water pooled into a raw water tank is suctioned and supplied to the tank body,

the vacuum suction step is a step wherein infectious waste water remaining in a raw water tank after the cleaning step or infectious waste water newly pooled into the raw water tank is vacuum-suctioned into the tank body which is rendered negative in pressure by the cleaning step,

the heating/sterilization step is a step wherein the heat of the steam fed into a heating part is passed through the wall surface of the tank body, allowed to act on infectious waste water, thus attaining sterilization of the infectious waste water,

the drainage step is a step wherein heated and sterilized waste water is discharged from the tank body, and

the water level formed by supplying infectious waste water into the tank body in the water supply step is positioned higher than the upper limit of the heating part which heats the tank body.

Further, in the system for sterilizing infectious waste water according to the invention, the infectious waste water supplied into the tank body is indirectly heated, and subjected to the heating/sterilization step while convecting inside the tank body, and a part of the tank body heated up to temperatures higher than drying temperatures of infectious waste water during the heating/sterilization step is submerged into the infectious waste water, thus preventing drying of solids contained in the infectious waste water.

In addition, the invention provides a system for sterilizing infectious waste water in a sterilization tank on the basis of indirect heating, wherein

the sterilization tank is provided with a steam heating means, a tank body and a drain pipe,

the steam heating means is to supply steam to a heating part formed on the outer wall of the tank body and allow the heat of the steam to indirectly act on the infectious waste water pooled into the tank body,

the tank body is a tank for receiving infectious waste water supplied by pump suction or vacuum suction due to negative pressure in the tank body, the erected height of the tank body is higher than the upper limit of the heating part and the water level of infectious waste water is constituted at a position higher than the upper limit of the heating part, and

the drain pipe is to release the treated waste water pooled into the tank body into the sewage system.

The invention further provides a system for sterilizing infectious waste water in a sterilization tank on the basis of indirect heating, having a pipeline cooler, wherein

the sterilization tank is provided with a tank body and a steam heating means,

the tank body is a tank for receiving infectious waste water supplied by pump suction or vacuum suction due to negative pressure in the tank body, to which the drain pipe is connected,

the steam heating means is to receive steam and allow the heat of the steam to indirectly act on infectious waste water pooled into the tank body,

the drain pipe is a pipe for releasing the sterilized and treated waste water pooled into the tank body into the sewage system,

the pipeline cooler is a pipe wherein externally-supplied cooling water is used to suction the treated waste water from the drain pipe, and is mixed with the waste water in the pipeline, and the mixed water is released into the sewage system.

Further, the abovementioned pipeline cooler is to eject cooling water supplied from a cooling-water supply source into the pipeline, producing negative pressure in the pipeline, thereby suctioning treated waste water forcibly from the tank body.

The abovementioned pipeline cooler is provided with a cooling-water supply source, a drain pipe and a pipeline connected to sewage pipe, and mixing cooling water supplied from the cooling-water supply source with treated waste water suctioned through the drain pipe to attain cooling inside the pipeline.

Further, the abovementioned pipeline cooler has a built-in nozzle, and the nozzle ejects cooling water supplied to the pipeline cooler at a high velocity to provide an ejector effect, thereby forcibly discharging waste water from the tank body into the drain pipe.

The abovementioned pipeline cooler is also provided with a cooling-water receiving port and a mixed-water feeding port for releasing waste water into the sewage system at both ends, and it is a pipe erected at a right angle in relation to the line connecting the cooling-water receiving port with the mixed-water feeding port, having a port for receiving treated waste water, a nozzle leading to the port for receiving cooling water is formed inside the pipe, a mixing chamber leading to the port for receiving treated waste water is formed at the front of the nozzle, the mixing chamber is provided with an opening reduced to a small diameter, and the opening leads to the port for feeding mixed-water.

The sewage pipe connected to the abovementioned pipeline cooler is provided with a U-shaped or L-shaped bent part at some point in the pipeline, and

the U-shaped or L-shaped bent part is to effectively mix cooling water supplied from the pipeline cooler with waste water discharged from the tank body.

### BRIEF DESCRIPTION OF THE DRAWINGS

FIG. 1(a) is a block diagram showing one embodiment of the invention and (b) is a view showing important parts.

FIG. 2 is a view illustrating one example of the sterilization tank.

FIG. 3(a) is an enlarged view of the sterilization tank used in the system of the invention, and (b) is an enlarged view of an ordinary sterilization tank.

FIG. 4(a) is a sectional view of the pipeline cooler used in the system of the invention, and (b) is a sectional view of line B-B in (a).

FIG. 5 is a flowchart showing steps according to the present invention.

FIG. 6 is a view illustrating a conventional example wherein waste water is pooled into a cooling tank connected to a sterilization tank to attain cooling.

### DETAILED DESCRIPTION OF THE PREFERRED EMBODIMENTS

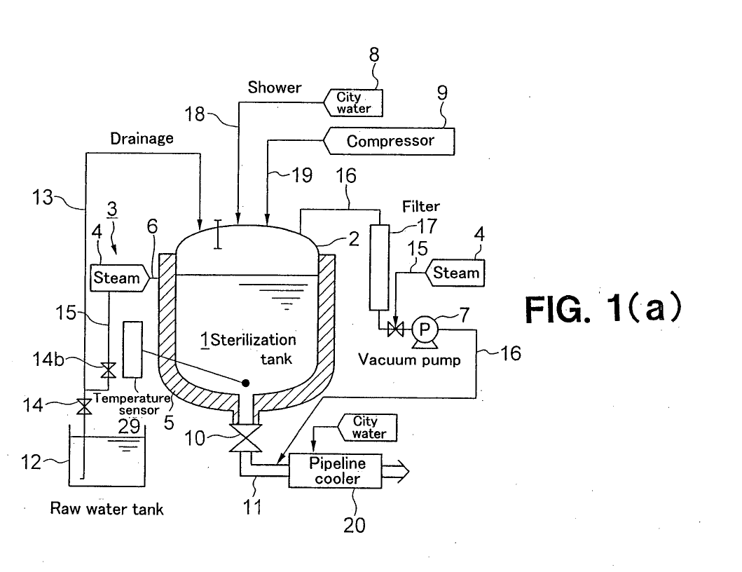

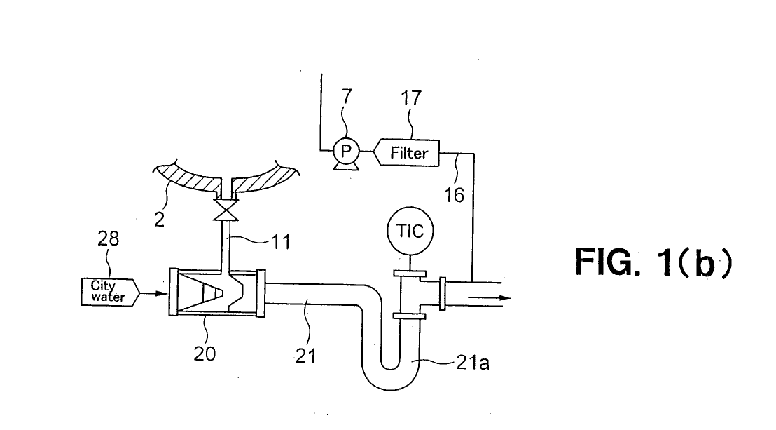

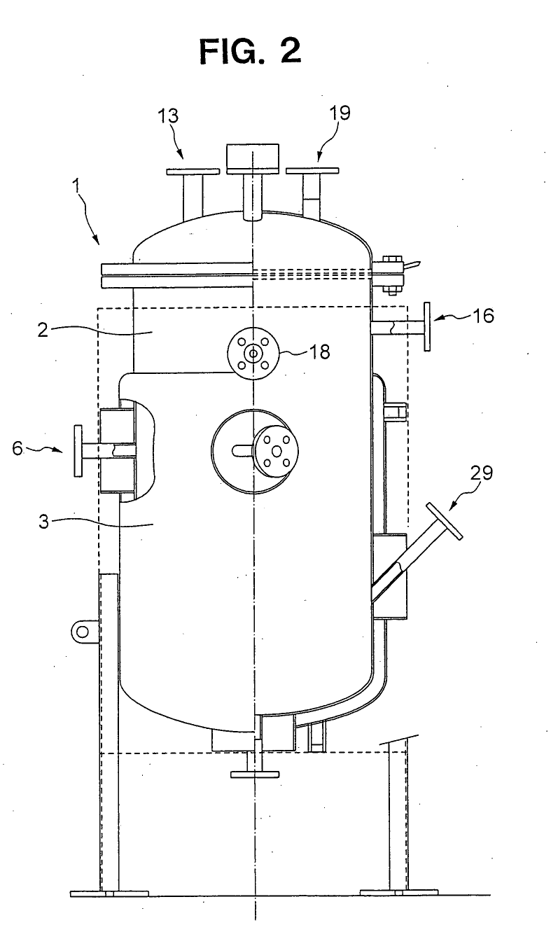

The embodiments of the invention will be explained hereinafter by referring to the figures. The system for sterilizing infectious waste water according to the invention is provided with a sterilization tank on the basis of indirect heating. In FIG. 1(a), the sterilization tank 1 is provided with the tank body 2 and the steam heating means 3. FIG. 2 shows an external appearance of the sterilization tank 1.

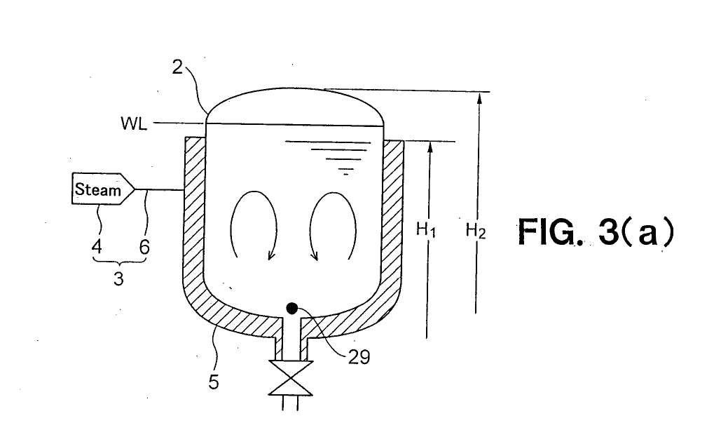

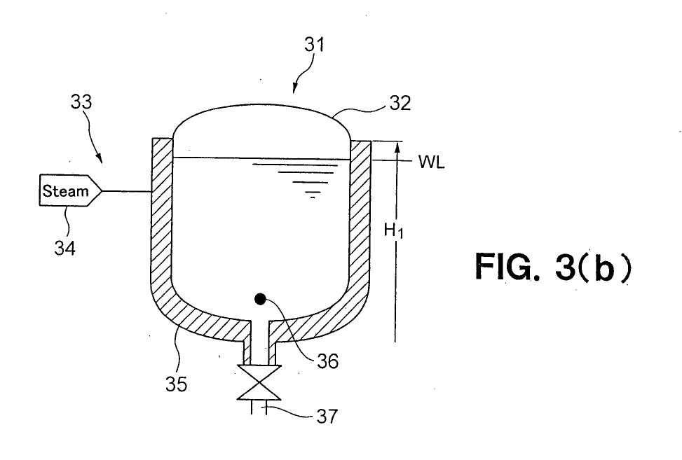

The tank body 2 is a tank for receiving infectious waste water to be sterilized, and the steam heating means 3 is mounted on the tank body 2 and provided with the heating part 5 for receiving steam produced by the steam generator 4. Steam produced by the steam generator 4 is fed into the heating part 5 through the steam pipe 6. The heating part 5 is, for example, a jacket assembled around the tank body 2, and infectious waste water pooled into the tank body 2 is heated indirectly by the heat of the steam conducted through the wall surface of the tank body 2 from the heating part 5. The tank body 2 shown in FIG. 1 is explained by an example where the water level of infectious waste water is constituted within a range of the height of the heating part 5 and the waste water is pooled into the tank body 2, as with the sterilization tank in a conventional sterilization system shown in FIG. 3(b). Therefore, when the waste water is heated with steam through the wall surface of the tank body 2, solids such as blood, protein, and fat are precipitated on the wall of the tank. In preventing precipitation of these solids, the sterilization tank shown in FIG. 3(a) is used.

In the sterilization tank shown in FIG. 3(a), the erected height H2 of the tank body 2 is made higher than the erected height H1 which is the upper limit of the heating part 5, (that is, H2>H1), and in contrast the erected height H1 of the heating part 5 is positioned lower than the water level WL of infectious waste water fed into the tank body 2. In preventing precipitation of the solids, it is preferable to have the structure of the sterilization tank as shown in FIG. 3(a). However, the sterilization tank may not be necessarily structured as shown in FIG. 3(a) in providing a method for sterilizing infectious waste water and the system thereof wherein negative pressure produced in the tank body after the cleaning step is utilized to suction infectious waste water pooled into a raw water tank into the tank body, thereby reducing running costs, and treated infectious waste water is cooled and released into the sewage system, without producing vibrations or noises.

Steam produced by the steam generator 4 is fed into the heating part 5 through the steam pipe 6, and infectious waste water pooled into the tank body 2 is indirectly heated by the heat of the steam. This process is needed also in the sterilization tank of FIG. 3(b) and in that of FIG. 3(a).

The tank body 2 is connected to the vacuum pump 7, the water release means 8 and the compressed air generator (compressor) 9 through the respective pipes, and the bottom of the tank body 2 is connected to the drain pipe 11 through the valve 10. Infectious waste water discharged from health care facilities such as hospitals is pooled into the raw water tank 12.

The tank body 2 and the raw water tank 12 are connected with the waste water supply pipe 13, and the waste water supply pipe 13 is provided with the valve 14 and connected at the downstream side (on the tank body side) with the steam pipe 15 leading to the steam generator 4 through the valve 14b.

The vacuum pump 7 is to cause deaeration in the tank body 2, thereby suctioning infectious waste water pooled into the raw water tank 12 to supply the waste water into the tank body 2. The vacuum pump 7 is connected to the tank body 2 through the pump pipe 16 and the pump pipe 16 is provided with the filter 17. The filter 17 is to catch bacteria contained in the suctioned air when air in the tank body 2 is suctioned. The pump pipe 16 is connected to the steam pipe 15 of the abovementioned steam generator 4, and steam sterilization is provided to the filter 17 including pump piping before the filter is exchanged. As will be explained later, after the cleaning step, negative pressure is produced inside the tank body 2, which can be utilized to vacuum-suction infectious waste water pooled into the raw water tank 12 into the tank body 2. Further, in suctioning the infectious waste water pooled into the raw water tank 12 to supply it into the tank body 2, a pressure pump may be used, other than a vacuum pump. The use of the vacuum pump is able to carry out vacuum suction inside the tank body, allowing the suction to act on infectious waste water pooled into a raw water tank to supply the waste water, without making the pump directly contact with the infectious waste water. The pressure pump is mounted on the pipe connecting the raw water tank 12 to the tank body. The pump is contaminated at the time of water supply by coming into contact with infectious waste water, but similar to the vacuum pump in the function for suctioning the infectious waste water pooled into the raw water tank 12 and supplying it to the tank body 2.

The water release means 8 is a shower. The shower mounted on the water pipe 18, which is a city water supply source, is provided in the tank body 2. The shower is for washing the inside of the tank body 2. The compressed air generator 9 is a compressor. The compressed air generator 9 is connected to the tank body 2 through the pressure application pipe 19. The compressed air generator 9 is to be used for removing foreign substances inside the waste water supply pipe 13.

The drain pipe 11 connected to the bottom of the tank body 2 is a pipe for releasing the waste water treated by heating sterilization and pooled into the tank body 2 into the sewage system. In this invention, it is for releasing the treated waste water from the drain pipe 11 through the pipeline cooler 20 into the sewage system.

The pipeline cooler 20 is a pipe wherein externally supplied cooling water is used to suction treated waste water from the drain pipe 11 and is also mixed with the treated waste water in the piping, thereby reducing the temperature to a fixed level so that the mixed water can be released into the sewage pipe 21.

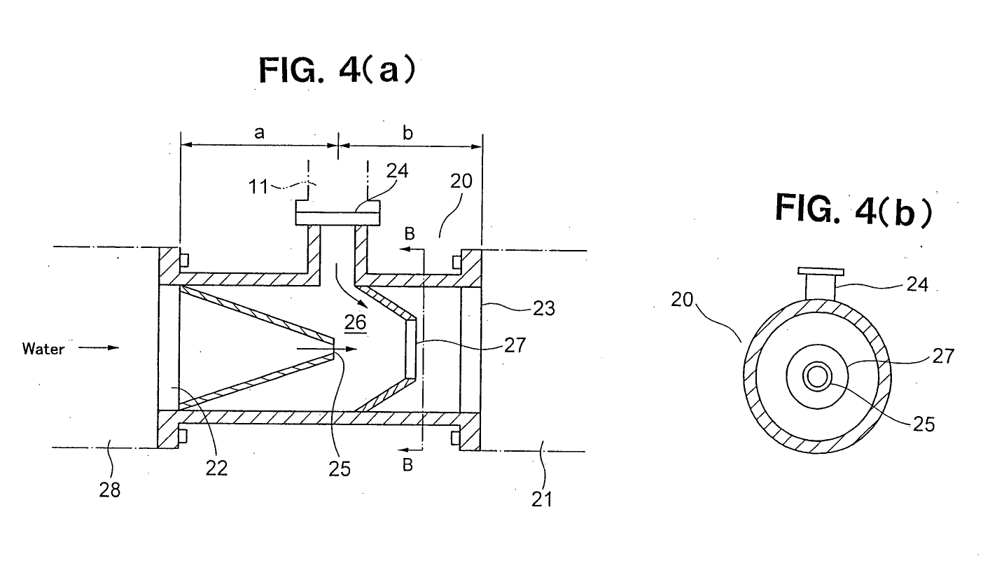

FIG. 4 shows the structure of the pipeline cooler 20. In FIG. 4, the pipeline cooler 20 is provided with a cooling-water receiving port 22 and a mixed-water feeding port 23 on both ends of the pipe. It is a three-way pipe erected at a right angle in relation to a line connecting the cooling-water receiving port 22 with the mixed-water feeding port 23 and provided with the treated waste water receiving port 24. In the pipe, a nozzle 25 is formed facing the cooling-water receiving port 22, a mixing chamber 26 is formed at front of the nozzle 25 facing the treated waste water receiving port 24, a small-diameter opening 27 is provided on the wall surface of the mixing chamber 26 facing the treated waste water receiving port 24 and the opening 27 leads to the mixed-water feeding port.

The treated waste water receiving port 24 is connected to the drain pipe 11 of the tank body 2. The cooling-water receiving port 22 and the mixed-water feeding port 23 are connected respectively to the city water supply source (water pipe) 28 and the sewage pipe 21. In FIG. 1, the numeral 29 denotes a temperature sensor. The temperature sensor 29 is mounted at the inner bottom of the tank body 2, and inserted from the outside of the tank body 2 at the bottom of the tank body 2, as shown in FIG. 2. The sewage pipe 21 is provided with a U-shaped bent part 21a at some point in the pipeline, as shown in FIG. 1(b). In FIG. 1(b), the U-shaped bent part 21a is provided at some point in the pipeline, but an L-shaped bent part (not illustrated) may be provided, in place of the U-shaped bent part.

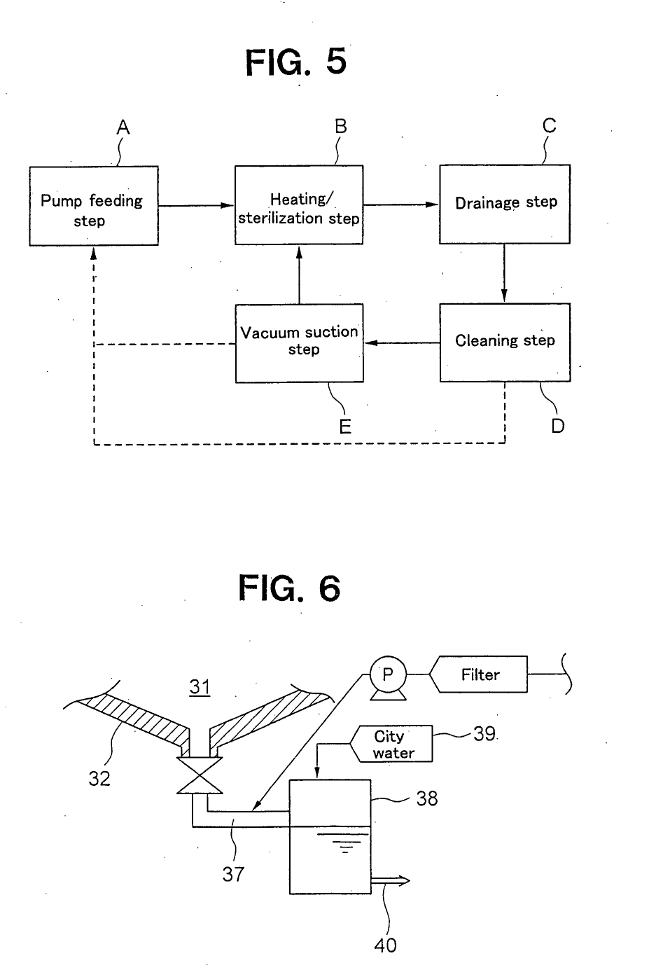

In this invention, infectious waste water discharged from a hospital is temporarily pooled into a raw water tank 12. The infectious waste water pooled into the raw water tank 12 is sterilized through various steps such as a water supply step, heating/sterilization step, drainage step and cleaning step. The water supply step shall mean a pump feeding step and vacuum suction step. The pump feeding step is a step wherein a vacuum pump or a pressure pump is used to suction the infectious waste water pooled into the raw water tank and supply it to the tank body. The vacuum suction step is a step wherein infectious waste water remaining in a raw water tank after the cleaning step or infectious waste water newly pooled into the raw water tank is vacuum-suctioned into the tank body which is rendered negative in pressure by the cleaning step, and used in place of the pump feeding step or in combination with the pump feeding step. FIG. 5 shows the flowchart of these steps.

In FIG. 5, the pump feeding step A is a step wherein suction by the pump 7 is provided to the tank body 2 of the sterilization tank 1 to supply the infectious waste water pooled into the raw water tank 12 to the tank body 2. When a vacuum pump 7 is used in the pump feeding step A, the vacuum pump 7 is started, with the valve 14 of the waste water supply pipe 13 closed, to effect deaeration inside the tank body 2 through the pump pipe 16. When a negative pressure higher than a fixed level is attained in the tank body 2, the vacuum pump 7 is stopped to open the valve 14 and infectious waste water pooled into the raw water tank 12 is then vacuum-suctioned into the waste water supply pipe 13 and fed into the tank body 2.

The heating/sterilization step B is a step wherein the heat of the steam is passed through the wall surface of the tank body 2 and allowed to act on the infectious waste water, thereby sterilizing the infectious waste water. Namely, steam produced by the steam generator 4 is transferred through the steam pipe 6 into the heating part 5. The heat of the steam transferred into the heating part 5 acts on the infectious waste water inside the tank body 2 through the wall surface of the tank body 2, thereby developing heat convection in the infectious waste water pooled into the tank body 2. Then, the infectious waste water is supplied all over to the tank body 2 by the heat of the steam to attain a uniform sterilization.

In the course of the heating/sterilization step B, the vacuum pump 7 is started again to effect deaeration inside the tank body 2, thereby attaining an improved sterilization effect.

The tank body 2 is rendered negative in pressure due to suction by the vacuum pump 7 but returned to positive pressure by the steam of waste water produced in the tank body 2. It is recommended to conduct sterilization under standard conditions, namely, at 121° C. to 132° C. for 20 minutes. Further, when needed, for example, at completion of daily work, steam produced by the steam generator 4 is introduced into the waste water supply pipe 13 and the pump pipe 16 to sterilize piping.

The drainage step C is a step wherein waste water treated by heating/sterilization is discharged from the tank body 2. In conducting the drainage step C, opening the valve 10 at the bottom of the tank body 2 after the heating/sterilization step makes it possible to utilize a positive pressure caused by the steam of the waste water, and high-pressure air produced by the compressed air generator 9 is introduced into the tank body 2 for supplementing a decreasing positive pressure along with advancement of the drainage step and a fixed level of pressure is provided to discharge the waste water.

The cleaning step D is a step wherein washing water is showered to the tank body 2 of the sterilization tank 1 after the drainage step to clean the tank body 2. In conducting the cleaning step D, city water is supplied to the emptied tank body 2 through the water supply pipe 18 of the water release means 8, thereby washing away foreign substances attached to the inner wall of the tank body 2 to clean the tank body 2.

The vacuum suction step E is a step wherein infectious waste water remaining in the raw water tank 12 after the cleaning step or that newly pooled into the raw water tank 12 is vacuum-suctioned into the tank body 2 which is rendered negative in pressure due to the cleaning step D without dependence on suction by the vacuum pump 7. The inside of the tank body 2 is heated to high temperatures (100° C. to 135° C.) by the heating/sterilization step and then cooled abruptly by city water showering released by the cleaning step after discharge. Experimental data is available wherein reduction of the vacuum degree of −0.01 to −0.04 MPa was attained for a tank with a capacity of 100 to 200 liters, depending on the capacity of the tank body.

In the vacuum suction step E, the tank body 2 is filled with infectious waste water vacuum-suctioned from the raw water tank 12. When the suction is not sufficiently potent, the vacuum pump 7 is used in combination or the vacuum pump 7 is started to switch to the pump feeding step by using a regular pump, and as described previously, the vacuum pump 7 is started, with the valve 14 of the waste water supply pipe 13 kept closed, to effect deaeration through the pump pipe 16 in the tank body 2. When a negative pressure higher than a fixed level is attained in the tank body 2, the vacuum pump 7 is stopped to open the valve 14, and infectious waste water pooled into the raw water tank 12 is suctioned into the tank body 2, and then steps are repeated such as heating/sterilization step B, drainage step C, cleaning step D and vacuum suction step E. The vacuum suction step E is a step for alleviating the load of the pump, and waste water may be freely supplied not by the vacuum suction step E but by pump feeding step A.

When infectious waste water is emptied from the raw water tank 12 by a series of steps, sterilization of the waste water will be completed at the last step of the cleaning step D. At completion of the sterilization step, a high-pressure air produced by the compressed air generator 9, with the valve 14 kept open, is introduced under pressure into the tank body 2, thereby allowing the high-pressure air introduced into the tank body 2 to flow reversely into the waste water supply pipe 13, thus washing away foreign substances, etc., remaining inside the waste water supply pipe 13 into the raw water tank 12 by the thus introduced high-pressure air.

On the other hand, city water is supplied as cooling water from the city-water supply source (city water pipe) 28 to the pipeline cooler 20 and mixed with the treated waste water, thereby reducing the temperature to less than a fixed level. In this invention, the cooling water supplied from the city water supply source is received at the cooling-water receiving port 22, squeezed by the nozzle 25 and ejected into the mixing chamber 26 at a higher pressure. Since a negative pressure is attained inside the mixing chamber 26 due to a high-pressure ejection of the cooling water, the treated waste water in the tank body 2 is forcibly suctioned by negative pressure inside the mixing chamber 26, fed into the pipeline cooler 20, mixed with the cooling water to reduce the temperature, then squeezed through the opening 27 on the mixing chamber 26, and the mixed water is passed through the mixed-water feeding port 23 and released into the sewage pipe 21.

A positive pressure is recovered inside the tank body 2 by the heating/sterilization step and a mere opening of the valve 10 allows waste water to discharge easily from the tank body 2 at first. However, such discharge becomes gradually difficult, with advancement of the drainage step, and may require pressure produced by utilizing high-pressure air of the compressed air generator 9 for transfer. In this instance, negative pressure inside the mixing chamber 26 can be utilized for discharging the waste water in the tank body 2, thereby reducing the energy required by the compressed air generator 9. On the other hand, mixed water flown into the sewage pipe 21 is passed through the U-shaped or L-shaped bent part 21a and improved for a mixture of waste water with the cooling water. Then, the mixed water is warmed (to about 40° C. to 45° C.) homogenously and released into the sewage system.

When high pressure air produced by the compressed air generator 9 is introduced under pressure into the tank body 2, with the valve 14 kept open, the high-pressure air introduced into the tank body 2 flows reversely inside the waste water supply pipe 13, thereby washing away foreign substances, etc., remaining in the waste water supply pipe 13 into the raw water tank 12 by the thus introduced high-pressure air.

As explained above, in conducting the first sterilization step of infectious waste water wherein the infectious waste water pooled into the raw water tank 12 is suctioned into the tank body 2 as a step of sterilization of infectious waste water, a series of pump feeding step A, heating/sterilization step B, drainage step C and cleaning step D are conducted in sequence. However, in the second sterilization step and thereafter wherein the infectious waste water pooled into the raw water tank 12 is suctioned into the tank body 2, the vacuum suction step E without the use of a pump can be utilized to relieve the load of the pump and also shorten the series of steps, thus carrying out the operation effectively.

Further, when the sterilization tank as shown in FIG. 3(a) is used, infectious waste water supplied to the tank body 2 is indirectly heated and subjected to the heating/sterilization step, while being convected inside the tank body 2, and the water level WL constituted by supplying the infectious waste water to the tank body 2 in the water supply step is positioned higher than the location of the erected height H1, an upper limit of the heating part 5 which heats the tank body 2, thus always keeping a high-temperature part of the tank body 2 heated at temperatures higher than the drying temperature of the infectious waste water during the step below the water level WL of the infectious waste water or into the infectious waste water, submerged into the waste water, thereby lessening precipitation of dried solids such as blood, protein, and fat contained in the infectious waste water and resulting in little chance that dried solids attach to the inner wall of the tank body 2.

It was considered that infectious waste water could not be heated up to a desired temperature even when the waste water was indirectly heated with steam, unless the infectious waste water was pooled into the tank body within a range of the erected height of the heating part. The sterilization tank was fabricated, in which the tank body as described in FIG. 3(a) was actually made with SUS316L and the heating part was made with SUS304, and the liquid temperature was determined to be 121° C. to 134° C. by using the temperature sensor 29 mounted at the inner bottom of the tank body 2.

Further, in this invention, city water supplied into the pipeline cooler 20 is passed through the nozzle 25 and ejected into the mixing chamber. The ejection water flow is used to suction treated waste water inside the tank body 2 and at the same time mix city water with high-temperature waste water, thereby preventing a phenomenon such as steam explosion found in a case where city water is fed into a huge amount of high-temperature infectious waste water, and the waste water undergoes expansion inside the mixing chamber by the energy produced upon mixture of high-temperature waste water with city water, but when the waste water is passed through the small-diameter opening 27 and flown into the side of the water feeding port, it undergoes shrinkage and interferential action by expansion and shrinkage to reduce the energy, thus effectively removing noises and vibrations.

In the experiment, the pipeline cooler was made with a 6 mm thick and 40 mm across SCS13 stainless foundry pipe. As shown in FIG. 4(a), an axis-center based distance between the cooling-water receiving port and the treated waste water receiving port and that between the mixed-water feeding port and the treated waste water receiving port were established to be a=110 mm and b=110 mm, respectively. The treated waste water receiving port 24 of the pipeline cooler 20 was connected to the drain pipe 11 on the tank body, and the mixed-water feeding port 23 was connected with the sewage pipe 21. Then, city water was supplied at 3 liters per second from the cooling-water receiving port 22, and a compressed air generator was started to discharge 1000 liter waste water pooled into the tank body 2 in order to conduct a cooling step.

The waste water in the tank body 2 was completely discharged in 0.5 (27.5 minutes) hours, during which no noise or vibration was reported. For comparison, as shown in FIG. 6, a stainless steel-made cooling tank 38 having a 230 liter internal volume and 160-liter effective impoundment was connected to the drain pipe 37 of the tank body 32, and city water was supplied at about 2 liters per second to the high-temperature treated waste water received from the tank body 32 into the cooling tank 38 to conduct cooling. Thereupon, loud discontinuous sounds were produced from the cooling tank 38 and small vibrations were detected at the cooling tank 38, drain pipe 37, and a connected part of the sewage pipe 40 connected to the cooling tank 38. Further, the compressed air generator was operated at a greatly reduced rate, as compared with a case where the cooling tank was used.

As explained so far, according to the invention, after the cleaning step, negative pressure developed in a tank body by the cleaning step is effectively utilized to vacuum-suction infectious waste water pooled into a raw water tank into the tank body, thus making it possible to shorten the series of steps without providing a load to a vacuum pump but reducing the operating rate, thereby attaining a reduction in running costs. Further, solids such as blood, protein, and fat contained in the infectious waste water in the tank body are precipitated less frequently on the inner wall, thus lessening the possibility of corroding the tank body and preventing problems such as clogging of piping due to falling of the solids. In addition, there is little chance of causing a steam explosion in discharging waste water, reducing noises and vibrations, and acting as a silencer for attaining a quiet operation. At the same time, the waste water is forcibly suctioned by utilizing an ejection energy of city water, thus greatly reducing the power of the compressed air generator used for feeding under pressure the waste water treated by heating/sterilization from the tank body.

In addition, according to the invention, the pipeline cooler is connected to the waste water pipeline to mix cooling water with the treated waste water while the waste water is circulating in the pipeline. Therefore, a U-shaped or L-shaped bent part is provided to a part of the sewage pipe, a downstream piping of the pipeline cooler, which allows the treated waste water and cooling water to be mixed more effectively through the bent part while running through the pipeline cooler down to the sewage pipe. In this instance, the mixed water is uniformly cooled and released to the sewage pipe.

What is claimed is:

1. A method for sterilizing infectious waste water, the method comprising steps of water supply, heating/sterilization, drainage, and cleaning, in which
the water supply step consists of a pump feeding step and vacuum suction step,
the pump feeding step is a step wherein the infectious waste water in a raw water tank is suctioned and supplied to a tank body,
the vacuum suction step is a step wherein infectious waste water remaining in the raw water tank after the cleaning step or infectious waste water newly pooled into the raw water tank is vacuum-suctioned into the tank body which is rendered negative in pressure by the cleaning step, and used in place of the pump feeding step or in combination with the pump feeding step,
the heating/sterilization step is a step wherein the heat of the steam is passed through the wall surface of the tank body, allowed to act on infectious waste water suctioned into the tank body of a sterilization tank, thus attaining sterilization of the infectious waste water,
the drainage step is a step wherein heated and sterilized waste water is discharged from the tank body, and
the cleaning step is a step wherein washing water is showered to the tank body of the sterilization tank after a drainage step to clean the tank body.

2. The method for sterilizing infectious waste water according to claim 1 wherein the pump feeding step is used when infectious waste water pooled into a raw water tank is suctioned for the first time to the tank body of a sterilization tank, and
the vacuum suction step is a step wherein after the pump feeding step, the heating/sterilization step, drainage step and cleaning step are carried out and then infectious waste water pooled into the raw water tank is suctioned into the tank body which is rendered negative in pressure by the cleaning step.

3. A method for sterilizing infectious waste water in a sterilization tank on the basis of indirect heating, the method comprising steps of water supply, heating/sterilization, and drainage, wherein
the sterilization tank consists of a tank body receiving infectious waste water and a heating part that steam-heats the tank body externally,
the water supply step is a step wherein infectious waste water is supplied into the tank body either by the pump feeding step or vacuum suction step,
the pump feeding step is a step wherein infectious waste water pooled into a raw water tank is suctioned and supplied to the tank body,
the vacuum suction step is a step wherein infectious waste water remaining in a raw water tank after the cleaning step or infectious waste water newly pooled into the raw water tank is vacuum-suctioned into the tank body which is rendered negative in pressure by the cleaning step,
the heating/sterilization step is a step wherein the heat of the steam fed into a heating part is passed through the wall surface of the tank body, allowed to act on infectious waste water, thus attaining sterilization of infectious wastewater,
the drainage step is a step wherein heated and sterilized waste water is discharged from the tank body, and
the water level formed by supplying infectious waste water into the tank body in the water supply step is positioned higher than the upper limit of the heating part which heats the tank body.

4. The method for sterilizing infectious waste water according to claim 3, wherein infectious waste water supplied into the tank body is indirectly heated, and subjected to the heating/sterilization step, while convecting inside the tank body, and a part of the tank body heated up to temperatures higher than the drying temperatures of infectious waste water during the heating/sterilization step is submerged into the infectious waste water, thus preventing drying of solids contained in the infectious waste water.

5. A system for sterilizing infectious waste water in a sterilization tank on the basis of indirect heating, wherein
the sterilization tank is provided with a steam heating means, a tank body and a drain pipe,
the steam heating means is to supply steam to a heating part formed on the outer wall of the tank body and allow the heat of the steam to indirectly act on infectious waste water pooled into the tank body,
the tank body is a tank for receiving infectious waste water supplied by pump suction or vacuum suction due to negative pressure in the tank body, the erected height of the tank body is higher than the upper limit of the heating part and the water level of infectious waste water is formed at a position higher than the upper limit of the heating part, and
the drain pipe is to release the treated waste water pooled into the tank body into a sewage system.

6. A system for sterilizing infectious waste water in a sterilization tank on the basis of indirect heating, having a pipeline cooler, wherein
the sterilization tank is provided with a tank body and a steam heating means,
the tank body is a tank for receiving infectious waste water supplied by pump suction or vacuum suction due to negative pressure in the tank body, to which the drain pipe is connected,
the steam heating means is to receive steam and allow the heat of the steam to indirectly act on infectious waste water pooled into the tank body,
the drain pipe is a pipe for releasing the sterilized and treated waste water pooled into the tank body into the sewage system,
the pipeline cooler is a pipe wherein externally-supplied cooling water is used to suction the treated waste water from the drain pipe and is mixed with the treated waste water in the pipeline, and the mixed water is released into the sewage system.

7. The system for sterilizing infectious waste water according to claim 6 wherein said pipeline cooler is to eject cooling water supplied from cooling-water supply source into the pipeline, producing negative pressure in the pipeline, thereby suctioning treated waste water forcibly from the tank body.

8. The system for sterilizing infectious waste water according to claim 6 wherein said pipeline cooler is provided with a cooling-water supply source, a drain pipe and a pipeline connected to a sewage pipe, and mixing cooling water supplied from the cooling-water supply source with treated waste water suctioned through the drain pipe to attain cooling inside the pipeline.

9. The system for sterilizing infectious waste water according to claim 6 wherein said pipeline cooler has a built-in nozzle, and
the nozzle ejects cooling water supplied to the pipeline cooler at a high velocity to provide an ejector effect, thereby forcibly discharging waste water from the tank body into the drain pipe.

10. The system for sterilizing infectious waste water according to claim 6 wherein said pipeline cooler is provided with a cooling-water receiving port and a mixed-water feeding port for releasing waste water into the sewage system at both ends, and is a pipe erected at a right angle in relation to the line connecting the cooling-water receiving port with the mixed-water feeding port, having a port for receiving treated waste water, a nozzle leading to the port for receiving cooling water is formed inside the pipe, a mixing chamber leading to the port for receiving treated waste water is formed at the front of the nozzle, a mixing chamber is provided with an opening reduced to a small diameter, and the opening leads to the port for feeding mixed-water.

11. The system for sterilizing infectious waste water according to claim 6 wherein the sewage pipe connected to said pipeline cooler is provided with a U-shaped or L-shaped bent part at some point in the pipeline, and
the U-shaped or L-shaped bent part is to effectively mix cooling water supplied from the pipeline cooler with waste water discharged from the tank body.
